(19) United States
(12) Patent Application Publication
(10) Pub. No.: US 2023/0377757 A1
(43) Pub. Date: Nov. 23, 2023

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Sho SASAKI, Utsunomiya (JP); Yudai YAMAZAKI, Nasushiobara (JP); Shinya SUGIYAMA, Nasushiobara (JP); Longxun PIAO, Nasushiobara (JP); Kosuke ARITA, Nasushiobara (JP)

(73) Assignee: CANON MEDICAL SYSTEMS CORPORATION, Otawara-shi (JP)

(21) Appl. No.: 18/318,018

(22) Filed: May 16, 2023

(30) Foreign Application Priority Data
May 20, 2022 (JP) 2022-082979

(51) Int. Cl. G16H 50/30

(52) U.S. Cl. CPC G16H 50/30

(57) ABSTRACT

A medical information processing device of embodiments includes processing circuitry. The processing circuitry is configured to acquire first time-series data regarding a first parameter and second time-series data regarding a second parameter different from the first parameter, identify a first risk range regarding the first parameter and a second risk range regarding the second parameter, and generate display information indicating the first time-series data and the second time-series data in association with each other on a display area in which the first risk range and the second risk range are associated.

### CROSS-REFERENCE TO RELATED APPLICATION

The present application claims priority based on Japanese Patent Application No. 2022-082979 filed May 20, 2022, the content of which is incorporated herein by reference.

### FIELD

Embodiments disclosed in this specification and drawings relate to a medical information processing device, a medical information processing method, and a storage medium.

### BACKGROUND

Conventionally, technology of presenting various time-series medical data such as examination values of patients and disease occurrence probabilities calculated using a machine learning model and the like to doctors is known as technology for supporting medical decision-making by doctors and the like. According to this technology, doctors can consider past and future circumstances of a patient and then make a treatment plan.

There are various types of medical data, and thus it is not possible to uniquely determine increase or decrease in each medical data value and a direction in which a risk that may occur to a patient increases or decreases. For example, as a value of disease occurrence probability data increases, a risk also increases. On the other hand, in case of blood pressure data, both excessive increase and excessive decrease in a value lead to risk increase. At the time of confirming a plurality of types of time-series medical data such as medical decision-making, doctors need to carefully discriminate increase/decrease and good/bad in risks for each piece of such medical data.

### BRIEF DESCRIPTION OF THE DRAWINGS

FIG. 1 is a diagram showing an example of a usage environment and functional blocks of a medical information processing device 1 according to a first embodiment.

FIG. 2 is a diagram showing an example of risk criterion information D1 according to the first embodiment.

FIG. 3 is a flowchart showing an example of a flow of processing of the medical information processing device 1 according to the first embodiment.

FIG. 4A is a diagram showing a heart failure occurrence probability (v-t) graph according to the first embodiment.

FIG. 4B is a diagram showing a heart failure occurrence probability (SR-t) graph according to the first embodiment.

FIG. 5A is a diagram showing a body weight (v-t) graph according to the first embodiment.

FIG. 5B is a diagram showing a body weight (SR-t) graph according to the first embodiment.

FIG. 6A is a diagram showing a motor function level (v-t) graph according to the first embodiment.

FIG. 6B is a diagram showing a motor function level (SR-t) graph according to the first embodiment.

FIG. 7A is a diagram showing a medicinal efficacy (v-t) graph according to the first embodiment.

FIG. 7B is a diagram showing a medicinal efficacy (SR-t) graph according to the first embodiment.

FIG. 8A is a diagram showing an LDL-C (v-t) graph according to the first embodiment.

FIG. 8B is a diagram showing an LDL-C (SR-t) graph according to the first embodiment.

FIG. 9A is a diagram showing another example of a body weight (v-t) graph according to the first embodiment.

FIG. 9B is a diagram showing another example of a body weight (SR-t) graph according to the first embodiment.

FIG. 10 is a diagram showing a superimposed graph displaying a plurality of types of medical data simultaneously according to the first embodiment.

FIG. 11 is a diagram showing a risk change trend (SR-c) graph according to a second embodiment.

FIG. 12A is a diagram showing a plurality of types of medical data (SR-t) graphs according to the second embodiment.

FIG. 12B is a diagram showing an example of a risk change trend (SR-c) graph according to the second embodiment.

FIG. 12C is a diagram showing another example of a risk change trend (SR-c) graph according to the second embodiment.

FIG. 12D is a diagram showing further another example of a risk change trend (SR-c) graph according to the second embodiment.

FIG. 13A is a diagram for describing vertex interpolation of a risk change trend (SR-c) graph according to the second embodiment.

FIG. 13B is a diagram for describing vertex interpolation of a risk change trend (SR-c) graph according to the second embodiment.

### DETAILED DESCRIPTION

A medical information processing device, a medical information processing method, and a storage medium according to embodiments will be described below with reference to the drawings.

A medical information processing device of embodiments includes processing circuitry. The processing circuitry is configured to acquire first time-series data regarding a first parameter and second time-series data regarding a second parameter different from the first parameter, identify a first risk range regarding the first parameter and a second risk range regarding the second parameter, and generate display information indicating the first time-series data and the second time-series data in association with each other on a display area in which the first risk range and the second risk range are associated.

### First Embodiment

A medical information processing device of a first embodiment makes it easier to check risks of a plurality of types of medical data and supports medical decision-making by standardizing the meanings of criteria of risks for the plurality of types of medical data (hereinafter referred to as “risk criteria”) and a direction of good or bad change in risks (hereinafter referred to as “risk change direction”). Risk criteria indicate a predetermined threshold ε, range θ, and the like for determining a risk of each piece of medical data. The threshold value ε indicates a clinical determination value such as a diagnostic threshold, a therapeutic threshold, or a preventive medicine threshold. The range θ indicates, for example, a criterion range or the like determined in advance in accordance with predetermined guidelines. A risk change direction indicates a direction of change in a medical data value with respect to risk criteria. The plurality of types of medical data include, for example, time-series data (examination values and the like) of an arbitrary patient, such as a heart failure occurrence probability, a body weight, a blood pressure, a motor function level, medicinal efficacy, LDL-C (bad cholesterol), and TG (triglycerides).

An increase in a risk of medical data means that the value of the medical data fluctuates in a direction in which a health condition becomes worse than health conditions within risk criteria. In the conventional method, when a risk criterion is defined by the threshold ε, it is determined that a risk has increased if a medical data value is greater than the threshold ε (or less than the threshold ε). On the other hand, when a risk criterion is defined by the range θ (β≥θ≥α), it is determined that a risk has increased if a medical data value is greater than the range θ (that is, the medical data value is greater than β) or if the medical data value is less than the range θ (that is, the medical data value is less than α). In this manner, it is assumed that a risk change direction differs according to the type of medical data in the conventional method. On the other hand, in the present embodiment, perspicuity with respect to various types of medical data is improved by standardizing the meaning of the risk change direction as a meaning that a fluctuation in a medical data value that becomes greater than a risk criterion is “increased risk.”

### [Configuration of Medical Information Processing Device]

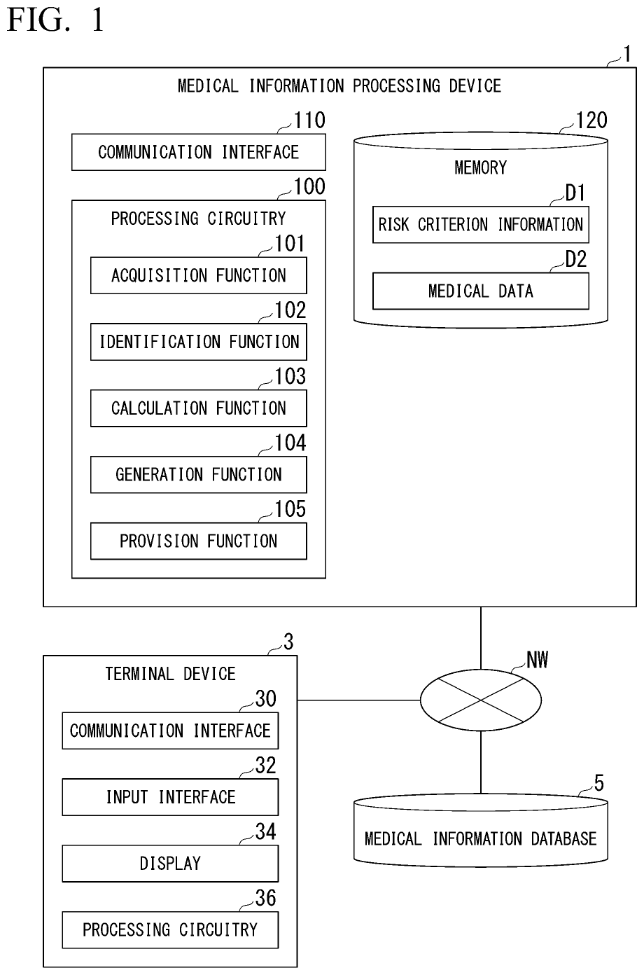

FIG. 1 is a diagram showing an example of a usage environment and functional blocks of a medical information processing device 1. The medical information processing device 1 is installed, for example, in a medical institution such as a hospital. The medical information processing device 1 may be, for example, a workstation, a server, or the like. The medical information processing device 1 is connected to, for example, at least one terminal device 3, at least one medical information database 5, and the like via a communication network NW such that data can be transmitted and received therebetween. The communication network NW indicates general information communication networks using telecommunication technology. The communication network NW includes a wireless/wired local area network (LAN) such as a hospital backbone LAN, an Internet network, a telephone communication network, an optical fiber communication network, a cable communication network, a satellite communication network, and the like.

The medical information processing device 1 includes, for example, processing circuitry 100, a communication interface 110, and a memory 120. The communication interface 110 communicates with external devices such as the terminal device 3 and the medical information database 5 via the communication network NW. The communication interface 110 includes, for example, a communication interface such as a network interface card (NIC).

The processing circuitry 100 includes, for example, an acquisition function 101, an identification function 102, a calculation function 103, a generation function 104, and a provision function 105. The processing circuitry 100 realizes these functions by, for example, a hardware processor (computer) executing a program stored in the memory 120 (storage circuit).

The hardware processor means, for example, circuit (circuitry) such as a central processing unit (CPU), a graphics processing unit (GPU), an application specific integrated circuit (ASIC), and a programmable logic device (for example, a simple programmable logic device (SPLD), a complex programmable logic device (CPLD), and a field programmable gate array (FPGA)). Instead of storing the program in the memory 120, the program may be configured to be directly incorporated into the circuit of the hardware processor. In this case, the hardware processor realizes the functions by reading and executing the program incorporated into the circuit.

The aforementioned program may be stored in the memory 120 in advance, or may be stored in a non-transitory storage medium such as a DVD or CD-ROM and installed in the memory 120 from the non-transitory storage medium when the non-transitory medium is set in a drive device (not shown) of the medical information processing device 1. The hardware processor is not limited to being configured as a single circuit, and may be configured as one hardware processor by combining a plurality of independent circuits to realize each function. Further, a plurality of components may be integrated into one hardware processor to realize each function.

The acquisition function 101 acquires a plurality of types of time-series medical data D2 from the medical information database 5 via the communication network NW and stores the same in the memory 120. The acquisition function 101 is an example of an “acquirer” in the scope of the claims. That is, the acquisition function 101 (acquirer) acquires first time-series data (first medical data) regarding a first parameter and second time-series data (second medical data) regarding a second parameter different from the first parameter.

The identification function 102 identifies a risk range of each of the acquired plurality of types of medical data D2 on the basis of risk criterion information D1 stored in the memory 120. Details of the risk range will be described later. The identification function 102 is an example of an “identifier” in the scope of the claims. That is, the identification function 102 (identifier) identifies a first risk range regarding the first parameter and a second risk range regarding the second parameter. The identification function 102 (identifier) identifies the first risk range and the second risk range on the basis of risk criteria preset according to the types of parameters.

The calculation function 103 calculates a standardized risk SR and a standardized threshold ST for each of the acquired plurality of types of medical data D2 on the basis of the risk criterion information D1 stored in the memory 120. A standardized risk SR is an index obtained by processing the value (v) of medical data using a conversion formula predetermined according to the type (pattern) of the medical data. Further, the standardized threshold ST is an index determined on the basis of the risk criterion information D1. Details of the standardized risk SR and the standardized threshold ST will be described later. The calculation function 103 is an example of a “calculator” in the scope of the claims. That is, the calculation function 103 (calculator) calculates a standardized risk and standardized threshold for each of the first time-series data and the second time-series data on the basis of risk criteria preset according to the types of parameters.

The generation function 104 generates display information indicating the acquired plurality of types of medical data D2 in association with each other using the calculated standardized risks SR and standardized thresholds ST. The generation function 104 generates, for example, a v-t graph in which the vertical axis represents a value (v) of medical data and the horizontal axis represents time (t). Furthermore, the generation function 104 generates, for example, an SR-t graph in which the vertical axis represents a standardized risk (SR), the horizontal axis represents time (t), and the origin (SR, t) is set to (ST, 0). Accordingly, different kinds of time-series medical data can be represented on one type of SR-t graph. In this SR-t graph, fluctuations in values greater than a criterion (standardized threshold ST) are standardized as a meaning of “increased risk.” The generation function 104 is an example of a “generator” in the scope of the claims. That is, the generation function 104 (generator) generates display information indicating the first time-series data and the second time-series data in association with each other on a display area in which the first risk range and the second risk range are associated. The generation function 104 (generator) generates display information indicating one standardized risk range by normalizing the first risk range and the second risk range. The generation function 104 (generator) associates the first risk range and the second risk range on the basis of the standardized threshold.

The provision function 105 transmits the generated display information to the terminal device 3 via the communication network NW.

The memory 120 is realized by, for example, a random access memory (RAM), a semiconductor memory device such as a flash memory, a hard disk, or an optical disk. These non-transitory storage media may be realized by other storage devices such as a network attached storage (NAS) and an external storage server device connected via the communication network NW. The memory 120 may also include non-transitory storage media such as a read only memory (ROM) and a register. The memory 120 stores, for example, the risk criterion information D1, the medical data D2, and the like. In addition, the memory 120 stores programs, parameter data, and other data used by the processing circuitry 100.

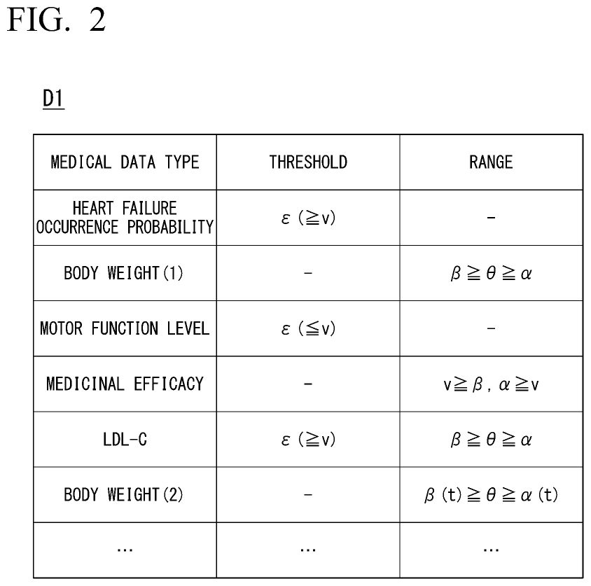

FIG. 2 is a diagram showing an example of the risk criterion information D1. As shown in FIG. 2, risk criteria (thresholds and ranges) are associated with types of medical data in the risk criterion information D1. For example, “threshold ε (≥v)” is associated with “heart failure occurrence probability” as a risk criterion. This threshold ε (≥v) means that, if the value (v) of the medical data is equal to or less than the threshold ε, the value is within a criterion range (good condition). Further, “range θ (β≥θ≥α)” is associated with “body weight” as a risk criterion, for example. This range θ (β≥θ≥α) means that, if the value (v) of the medical data is within the range θ (β≥v≥α), the value is within the criterion range (good condition). Further, “threshold ε (≥v)” and “range θ (β≥θ≥α)” are associated with “LDL-C” as risk criteria, for example.

### [Configuration of Terminal Device 3]

The terminal device 3 is a device for referring to display information (medical data) provided by the medical information processing device 1. The terminal device 3 is operated by an operator such as a doctor or a technician, for example. The terminal device 3 is, for example, a personal computer, a mobile terminal such as a tablet or a smartphone.

The terminal device 3 includes, for example, a communication interface 30, an input interface 32, a display 34, and processing circuitry 36. The communication interface 30 communicates with external devices such as the medical information processing device 1 and the medical information database 5 via the communication network NW.

The input interface 32 receives various input operations from the operator of the terminal device 3, converts the received input operations into electrical signals, and outputs the electrical signals to the processing circuitry 36. For example, the input interface 32 includes a mouse, a keyboard, a trackball, a switch, a button, a joystick, a touch panel, and the like. The input interface 32 may be, for example, a user interface that receives voice input, such as a microphone.

The input interface in this specification is not limited to those having physical operation parts such as a mouse and a keyboard. For example, the input interface includes electrical signal processing circuitry that receives an electrical signal corresponding to an input operation from an external input device provided separately from the apparatus and outputs the electrical signal to a control circuit.

The display 34 displays various types of information. For example, the display 34 displays an image generated by the processing circuitry 36, a graphical user interface (GUI) for receiving various input operations from the operator, and the like. For example, the display 34 is a liquid crystal display (LCD), a cathode ray tube (CRT) display, an organic electroluminescence (EL) display, or the like.

The processing circuitry 36 activates a dedicated application program, browser, or the like and causes the display 34 to display display information (medical data) provided by the medical information processing device 1. Further, the processing circuitry 36 generates a GUI for receiving various input operations from the operator and causes the display 34 to display the GUI. For example, the processing circuitry 36 generates a GUI for receiving an input operation requesting medical data from the operator, causes the display 34 to display the GUI, and when an input operation requesting acquisition of medical data is received, transmits a medical data acquisition request to the medical information processing device 1.

### [Configuration of Medical Information Database 5]

The medical information database 5 stores medical data for each patient acquired by various diagnostic devices, examination devices, and the like. The medical information database 5 stores, for example, arbitrary medical data such as heart failure occurrence probabilities, body weights, blood pressures, motor function levels, medicinal efficacy, LDL-C (bad cholesterol), TG (triglycerides), and the like associated with patient IDs. The medical information database 5 is realized by, for example, a RAM, a semiconductor memory device such as a flash memory, a hard disk, or an optical disk. The medical information database 5 may be incorporated in the medical information processing device 1.

### [Processing Flow]

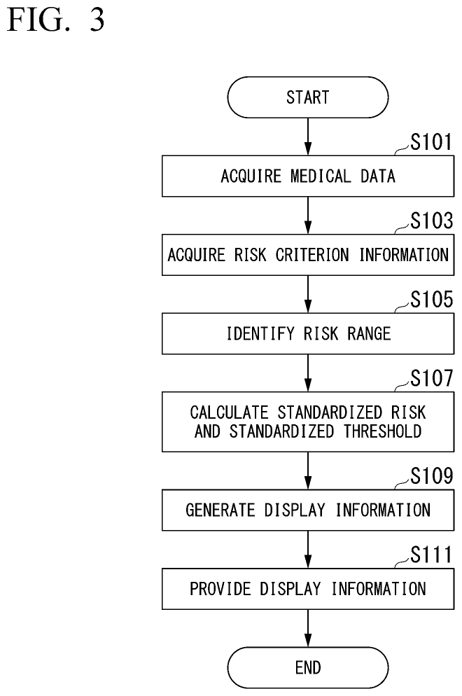

Next, processing of the medical information processing device 1 will be described. FIG. 3 is a flowchart showing an example of a flow of processing of the medical information processing device 1. Processing shown in FIG. 3 is executed, for example, when the medical information processing device 1 receives a processing request transmitted from the terminal device 3 on the basis of an operation of a doctor or the like.

First, the acquisition function 101 acquires a plurality of types of time-series medical data of a patient that is an acquisition target from the medical information database 5 via the communication interface 110 in response to an acquisition request from the terminal device 3 (step S101). The acquisition function 101 acquires the medical data from the medical information database 5 on the basis of a patient ID included in the acquisition request, for example. The acquisition function 101 stores the acquired medical data in the memory 120.

Next, the identification function 102 acquires the risk criterion information D1 from the memory 120 (step S103). The identification function 102 acquires, for example, information on risk criteria (thresholds and/or ranges) corresponding to the types of medical data acquired by the acquisition function 101.

Next, the identification function 102 identifies a risk range for each type of medical data on the basis of the risk criteria (step S105). Next, the calculation function 103 calculates a standardized risk SR and a standardized threshold ST on the basis of the risk criteria and the medical data (step S107). Details of processing of identifying a risk range and processing of calculating a standardized risk SR and a standardized threshold ST differ depending on types of medical data (settings of risk criteria). Such processing will be described below for each setting of risk criteria.

(1) Case in which Threshold ε (≥v) is Set as Risk Criterion

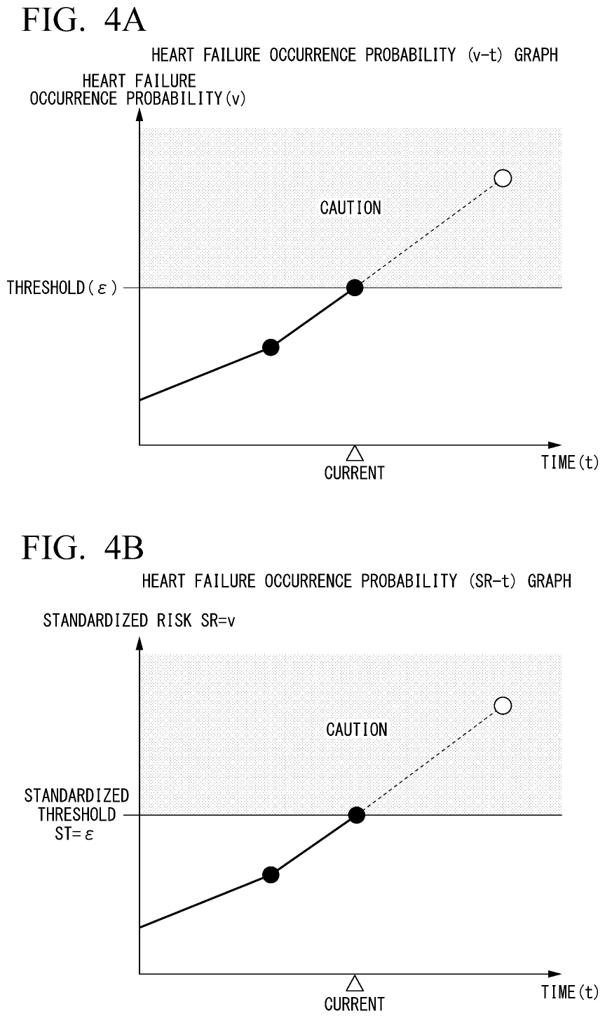

FIGS. 4A and 4B are diagrams showing a heart failure occurrence probability (v-t) graph and (SR-t) graph. As shown in FIG. 2, only a threshold is defined as a risk criterion for the heart failure occurrence probability. Furthermore, this threshold is “ε (≥v),” that is, a fluctuation in a medical data value that becomes greater than the criterion threshold ε is a pattern of increased risk. In this case, as shown in FIG. 4A, when the horizontal axis is time (t) and the vertical axis is heart failure occurrence probability (v) (medical data value), the identification function 102 identifies a range exceeding the threshold ε on the vertical axis as a risk range (range of caution).

Further, as shown in FIG. 4B, the calculation function 103 calculates the same value as the heart failure occurrence probability (v) as a standardized risk SR (SR=v). In addition, the calculation function 103 calculates the same value as the threshold ε as a standardized threshold ST (ST=ε). As a result, the heart failure occurrence probability (SR-t) graph shown in FIG. 4B has the same waveform as the heart failure occurrence probability (v-t) graph shown in FIG. 4A. The black circles shown in FIGS. 4A and 4B indicate measured values, and the white circles indicate predicted values. The same applies to other graphs below.

That is, when a threshold is set as a risk criterion, the calculation function 103 (calculator) sets the threshold as a standardized threshold and calculates the values of the first time-series data and the second time-series data as a standardized risk.

(2) Case in which Range (β≥δ≥α) is Defined as Risk Criterion

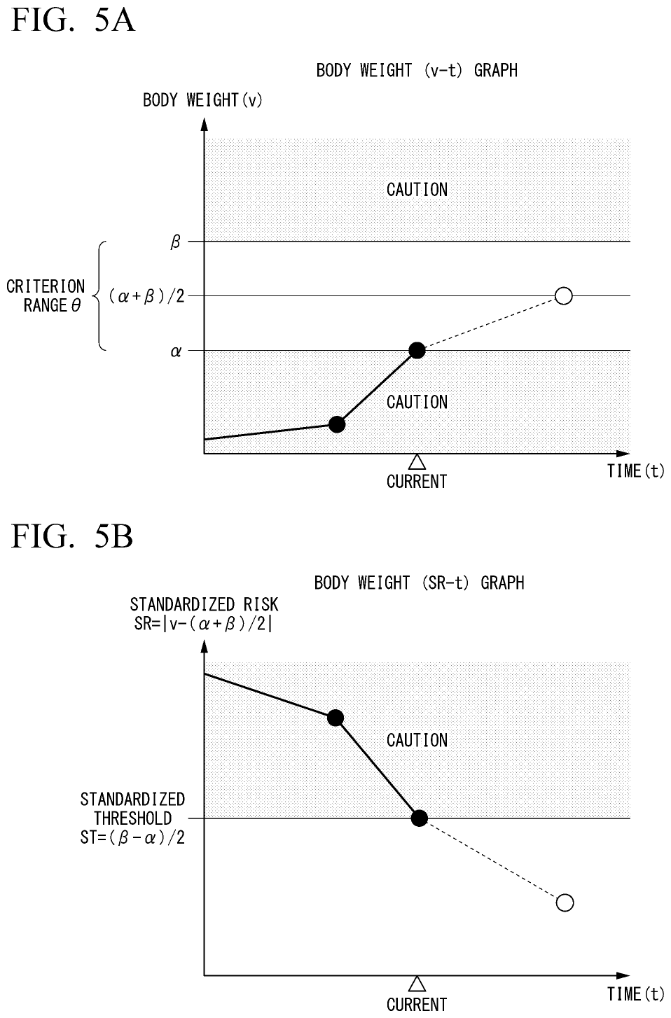

FIGS. 5A and 5B are diagrams showing a body weight (v-t) graph and (SR-t) graph. As shown in FIG. 2, only a range is defined as a risk criterion for body weight (1). Furthermore, this range is “β≥θ≥α,” that is, both a fluctuation (v>θ) in a medical data value that becomes greater than the criterion (range θ) and a fluctuation (v<θ) in the medical data value that becomes less than the risk criterion are patterns of increased risk. This is because both excessive increase and excessive decrease in a body weight lead to risk increase. In this case, as shown in FIG. 5A, when the horizontal axis is time (t) and the vertical axis is body weight (v), the identification function 102 identifies both a range excluding the criterion range θ on the vertical axis, that is, a range exceeding β and a range below α as risk ranges (ranges of caution).

Furthermore, as shown in FIG. 5B, the calculation function 103 calculates a standardized risk SR on the basis of the following formula (1). In addition, the calculation function 103 calculates a standardized threshold ST on the basis of the following formula (2). As a result, in the body weight (SR-t) graph shown in FIG. 5B, fluctuations in body weight values that become greater than the criterion (standardized threshold ST) are standardized as a meaning of “increased risk.”

SR=|v−(α+β)/2| Formula (1)

ST=(β−α)/2 Formula (2)

That is, when a range is set as a risk criterion, the calculation function 103 (calculator) calculates a standardized risk on the basis of a midpoint value between the upper limit value and the lower limit value of the range. When the identifier identifies two risk ranges for one parameter on the basis of a risk criterion, the calculation function 103 (calculator) sets a value corresponding to half the difference between the upper limit value and the lower limit value of the ranges to a standardized threshold.

(3) Case in which Threshold ε (≤v) is Defined as Risk Criterion

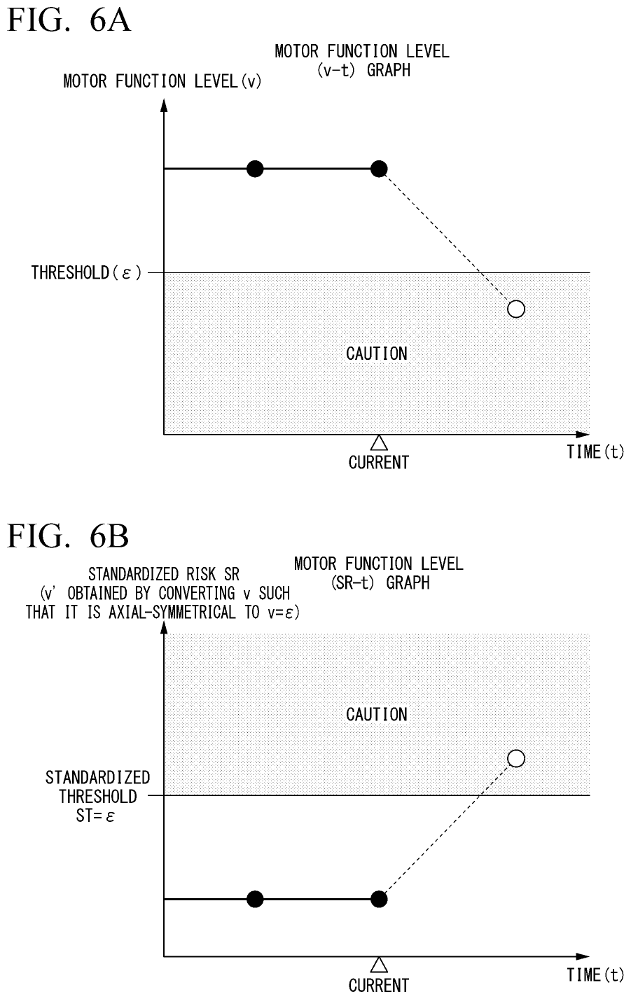

FIGS. 6A and 6B are diagrams showing a motor function level (v-t) graph and (SR-t) graph. As shown in FIG. 2, only a threshold is defined as a risk criterion for motor function levels. Furthermore, this threshold is “ε(≤v)”, that is, a fluctuation in a medical data value that becomes less than the risk criterion is a pattern of increased risk. In this case, as shown in FIG. 6A, when the horizontal axis is time (t) and the vertical axis is a motor function level (v), the identification function 102 identifies a range below the threshold ε on the vertical axis as a risk range (range of caution).

Furthermore, as shown in FIG. 6B, the calculation function 103 calculates v obtained by converting the motor function level (v) such that it is axial-symmetrical to v=ε as a standardized risk SR (SR=v′). In addition, the calculation function 103 calculates the same value as the threshold ε as a standardized threshold ST (ST=ε). As a result, the motor function level (SR-t) graph shown in FIG. 6B has a waveform obtained by converting the motor function level (v-t) graph shown in FIG. 6A such that it is axial-symmetrical to v=ε, and fluctuations in values that become greater than the criterion (standardized threshold ST) are standardized as a meaning of “increased risk.”

That is, when a threshold is set as a risk criterion, the calculation function 103 (calculator) sets the threshold as a standardized threshold and calculates values obtained by symmetrically converting the values of the first time-series data and the second time-series data on the basis of the threshold as a standardized risk.

(4) Case in which Ranges (v≥β, α≥v) are Defined as Risk Criterion

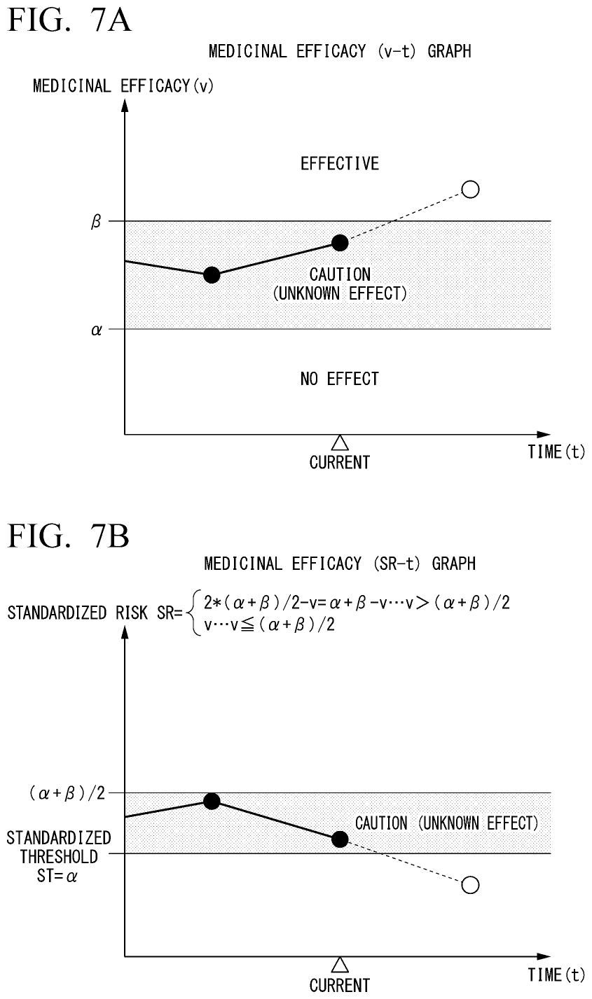

FIGS. 7A and 7B are diagrams showing a medicinal efficacy (v-t) graph and (SR-t) graph. As shown in FIG. 2, only ranges are defined as risk criteria for medicinal efficacy. Further, these ranges are “v≥β and α≥v,” that is, medical data included in a predetermined range has a pattern of increased risk. A range in which it can be determined that there is medicinal efficacy (v≥β) and a range other than a range in which it can be determined that there is no medicinal efficacy (α≤v), that is, a range of unknown medicinal efficacy in which it cannot be determined that there is medicinal efficacy and there is no medicinal efficacy leads to risk increases. In this case, as shown in FIG. 7A, when the horizontal axis is time (t) and the vertical axis is medicinal efficacy (v), the identification function 102 identifies a range “β>v>α” on the vertical axis as a risk range (range of caution).

Furthermore, as shown in FIG. 7B, the calculation function 103 calculates a standardized risk SR on the basis of the following formulas (3) and (4). In addition, the calculation function 103 calculates a standardized threshold ST on the basis of the following formula (5). As a result, in the medicinal efficacy (SR-t) graph shown in FIG. 7B, fluctuations in medicinal efficacy that become greater than the risk criterion (standardized threshold ST) are standardized as a meaning of “increased risk.”

SR=2*(α+β)/2−v=α+β−v . . . v>(α+β)/2 Formula (3)

SR=v . . . v≤(α+β)/2 Formula (4)

ST=α Formula (5)

That is, when the identifier identifies one risk range for one parameter on the basis of the risk criteria, the calculation function 103 (calculator) calculates the lower limit value of the range as a standardized threshold.

(5) Case in which Both Threshold and Range are Defined as Risk Criteria

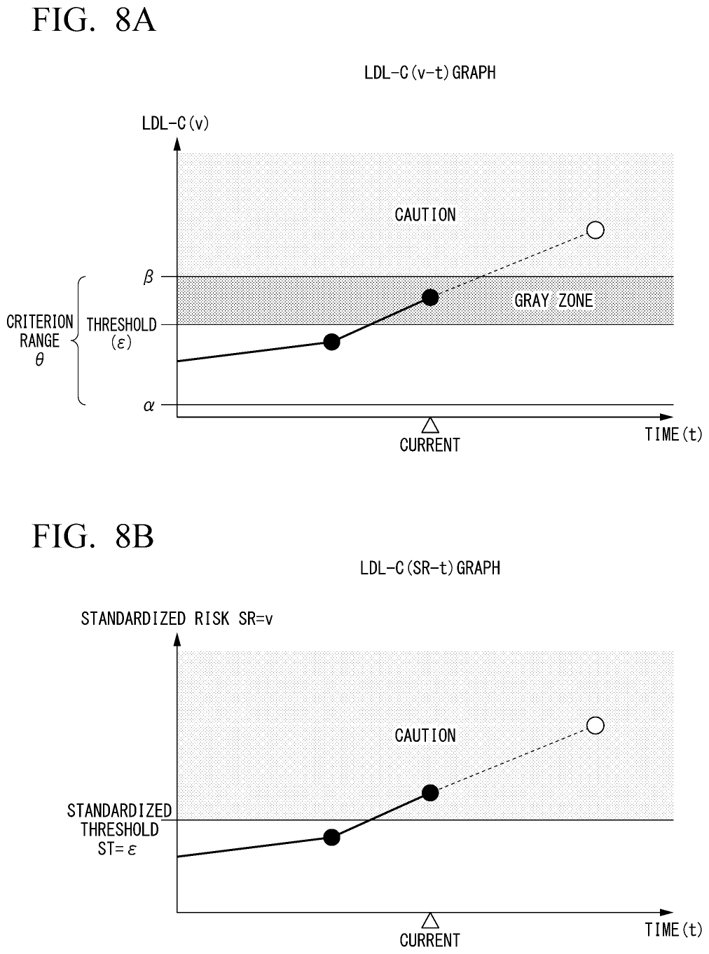

FIGS. 8A and 8B are diagrams showing an LDL-C (v-t) graph and (SR-t) graph. As shown in FIG. 2, both a threshold and a range are defined as risk criteria for LDL-C. Here, the threshold “ε(≥v)” is set within the range “β≥θ≥α.” Thus, there are cases in which a clinical determination value (diagnostic threshold (ε)) lower than the criterion range upper limit value (0) is set for LDL-C. In this case, as shown in FIG. 8A, when the horizontal axis is time (t) and the vertical axis is LDL-C (v), the identification function 102 defines an area that exceeds the threshold ε on the vertical axis but is within the range “β≥θ≥α” as a gray zone (quasi-risk range) and identifies an area other than the range “β≥θ≥α” as a range of caution.

Further, as shown in FIG. 8B, the calculation function 103 calculates the same value as LDL-C(v) as a standardized risk SR (SR=v). In addition, the calculation function 103 calculates the same value as the threshold ε as a standardized threshold ST (ST=ε). As a result, the LDL-C (SR-t) graph shown in FIG. 8B has the same waveform as the LDL-C (v-t) graph shown in FIG. 8A, and fluctuations in values that become greater than the criterion (standardized threshold ST) are standardized as a meaning of “increased risk.”

In addition, there is an example in which both a threshold and a range are defined as risk criteria for TG (triglycerides). With respect to this TG, a clinical determination value (diagnostic threshold) higher than a criterion range upper limit value may be set. In this case, the lower value between the diagnostic threshold and the criterion range upper limit value (in this case, the criterion range upper limit value) may be set as a standardized threshold ST. At this time, display may be performed such that inclusion of a gray zone can be ascertained.

That is, when both a threshold and a range are set for one parameter as risk criteria, the identification function 102 (identifier) identifies a quasi-risk range on the basis of the threshold and the range. The calculation function 103 (calculator) calculates the smaller value between the threshold and the lower limit value of the risk range of one parameter as a standardized threshold.

(6) Case in which Non-Linear Threshold or Range is Defined as Risk Criterion

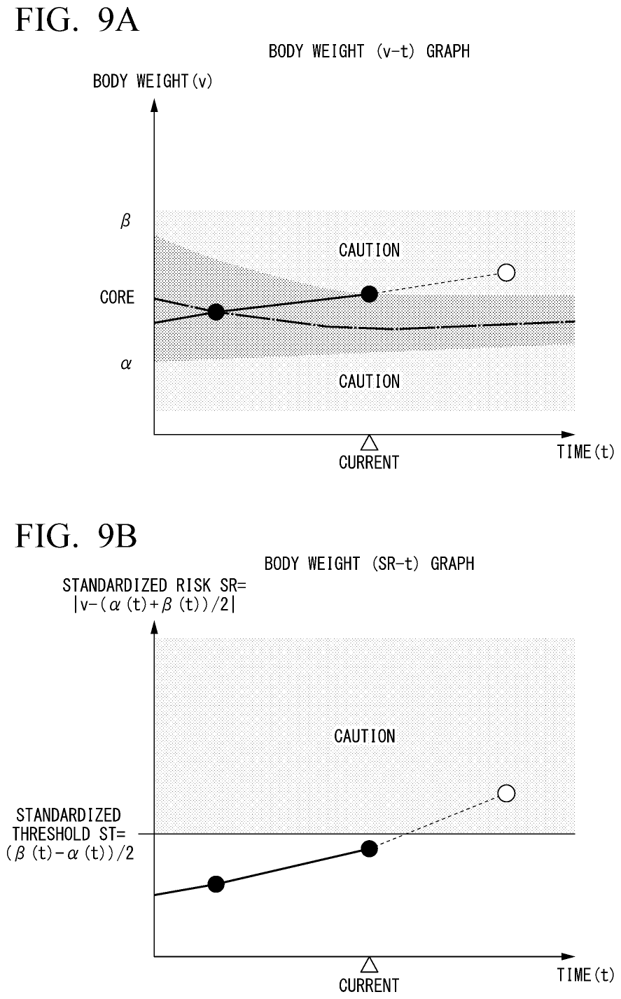

FIGS. 9A and 9B are diagrams showing other examples of a body weight (v-t) graph and (SR-t) graph. As shown in FIG. 2, only a range is defined as a risk criterion for body weight (2). Further, this range is “β(t)≥θ≥α(t),” that is, the definition of the range of the risk criterion is a function (non-linear) of time (t). In this case, as shown in FIG. 9A, when the horizontal axis is time (t) and the vertical axis is body weight (v), the identification function 102 identifies both a range excluding a criterion range θ on the vertical axis, that is, a range exceeding β(t) and a range below α(t) as risk ranges (ranges of caution).

Further, as shown in FIG. 9B, the calculation function 103 calculates a standardized risk SR on the basis of the following formula (6). The calculation function 103 also calculates a standardized threshold ST on the basis of the following equation (7). As a result, in the body weight (SR-t) graph shown in FIG. 9B, fluctuations in a body weight value that become greater than the criterion (standardized threshold ST) is standardized as a meaning of “increased risk.”

SR=|v−(α(t)+β(t))/2| Formula (6)

ST=(β(t)−α(t))/2 Formula (7)

An example of a case in which, at the time of determining a standardized threshold ST of a range, the midpoint of the range is set as a standardized threshold ST on the assumption that medical data conforms to a normal distribution has been described above. If medical data does not conform to a normal distribution, the threshold of the range may be designated as the midpoint by transforming the data to a normal distribution using a parametric method.

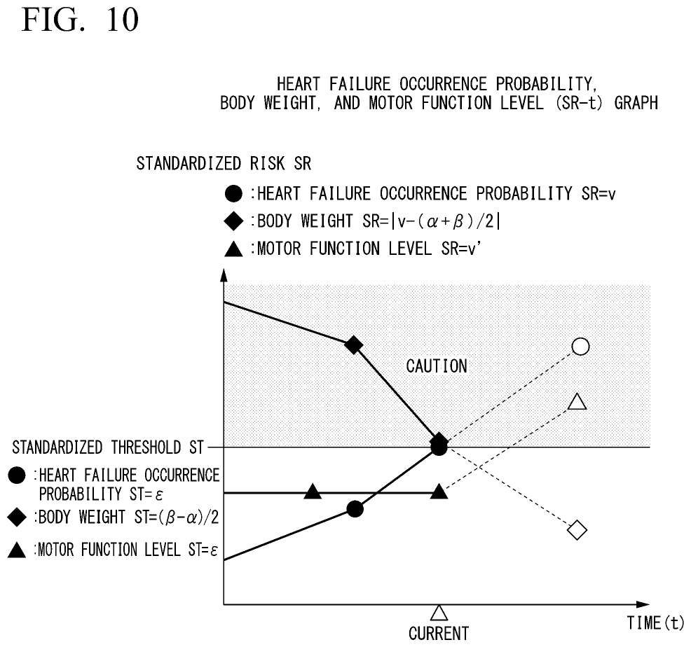

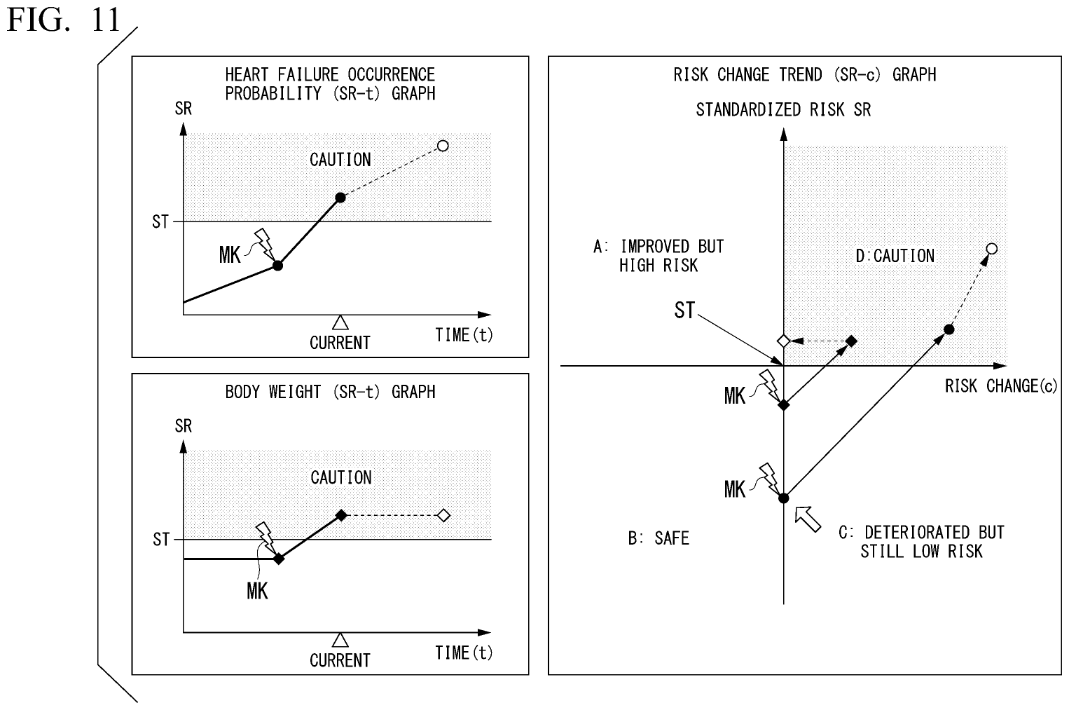

Referring back to FIG. 3, the generation function 104 generates display information indicating the plurality of types of medical data D2 acquired by the acquisition function 101 in association with each other using the standardized risk SR and the standardized threshold ST calculated by the calculation function 103 (step S109). FIG. 10 is a diagram showing a superimposed graph in which a plurality of types of medical data are superimposed and displayed simultaneously. The superimposed graph shown in FIG. 10 shows the heart failure occurrence probability (SR-t) graph shown in FIG. 4B, the body weight (SR-t) graph shown in FIG. 5B, and the motor function level (SR-t) graph shown in FIG. 6B in a superimposed manner by aligning the positions of the standardized thresholds ST. As described above, fluctuations in values that become greater than the criteria (standardized thresholds ST) are standardized as a meaning of “increased risk” in the graphs shown in FIG. 4B, FIG. 5B, and FIG. 6B, and thus it is possible to display such a superimposed graph shown in FIG. 10. A doctor can refer to this superimposed graph and easily check risks of the plurality of types of medical data simultaneously. For example, a doctor can make medical decision by focusing on medical data more deeply included in a range of caution.

That is, the generation function 104 (generator) generates display information that is a graph in which the horizontal axis represents time and the vertical axis represents a standardized risk.

Normalization processing may be performed on an SR-t graph such that a standardized threshold ST is set to 0.5 and a standardized risk SR falls within the range of 0 to 1. Accordingly, it is possible to improve the visibility at the time of displaying a plurality of types of medical data on the superimposed graph shown in FIG. 10. Further, the vertical axis (standardized risk SR axis) may be displayed in logarithm.

In addition, related data among the plurality of types of medical data may be displayed such that the fact that they are related can be ascertained on the superimposed graph shown in FIG. 10. For example, at the time of simultaneously displaying data of “left ventricular ejection fraction (LVEF)” and “brain natriuretic peptide (BNP)” related to the disease of heart failure, they may be displayed in colors close to each other, and when one data is selected, the other data may be highlighted. In addition, the data may be displayed such that risks up to the present and future risks can be distinguished. For example, risks up to the present may be displayed using a solid line, and future risks may be displayed using a dashed line.

Next, the provision function 105 transmits (provides) the display information generated by the generation function 104 to the terminal device 3 via the network NW (step S111). Accordingly, the display information is displayed on the display of the terminal device 3, and the doctor can check the display information. In this manner, processing of this flowchart ends. Although continuous type data has been described as an example above, the present invention can also be applied to discrete type data such as the number of medicine administrations.

According to the first embodiment described above, it is possible to easily check risks of a plurality of types of medical data and support medical decision-making. Accordingly, among the plurality of types of medical data, data to be focused can become obvious. In addition, oversight of various risk changes of patients can be reduced.

### Second Embodiment

A second embodiment will be described below. The second embodiment differs from the first embodiment in that the medical information processing device 1 generates, as display information, a risk change trend (SR-c) graph showing the amount of change in risk per unit time instead of the superimposed graph (SR-t graph). Hereinafter differences from the first embodiment will be mainly described, and description of common points with the first embodiment will be omitted. In the description of the second embodiment, the same parts as those in the first embodiment are denoted by the same reference numerals.

FIG. 11 is a diagram showing a risk change trend (SR-c) graph. The graph shown in FIG. 11 is generated on the basis of the heart failure occurrence probability (SR-t) graph shown in FIG. 4B and the body weight (SR-t) graph shown in FIG. 5B. Alternatively, the graph shown in FIG. 11 is generated on the basis of the heart failure occurrence probability (v-t) graph shown in FIG. 4A and the body weight (v-t) graph shown in FIG. 5B. In the graph shown in FIG. 11, the horizontal axis represents risk change (c) indicating the amount of change in risk per unit time, and the vertical axis represents a standardized risk SR. The risk change (c) is calculated by the following formula (8).

Risk change c=v(t)−v(t−1) Formula (8)

In the graph shown in FIG. 11, a first quadrant (D: caution), a second quadrant (A: improved but high risk), a third quadrant (B: safe), and a fourth quadrant (C: deteriorated but still low risk) are defined. In addition, the origin (SR, c) is set to (ST, 0). Further, a mark MK attached to the standardized risk SR of each piece of medical data indicates that times (t) of vertices (plots) correspond to each other. A doctor can easily check the amount of change in risk per unit time by checking this risk change trend (SR-c) graph. In particular, the doctor can make medical decision by focusing on medical data in which greater risk change has occurred.

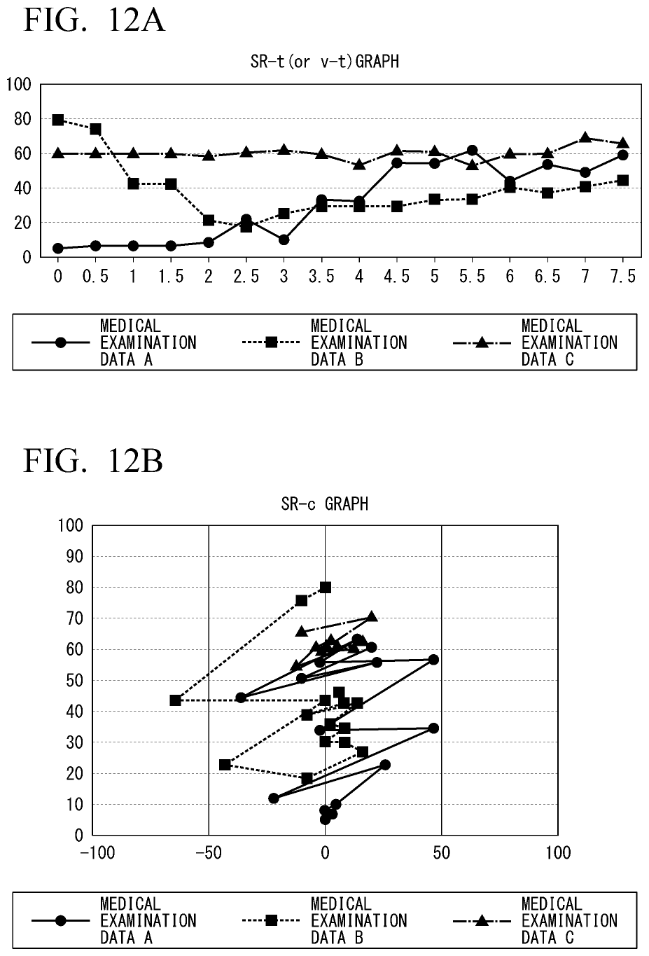

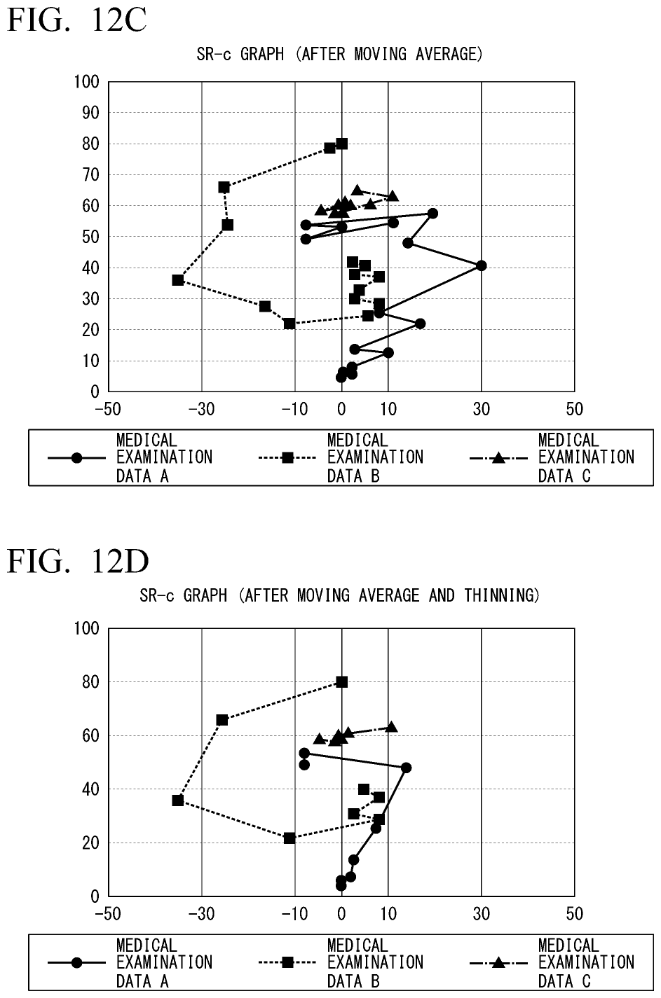

FIGS. 12A to 12D are diagrams showing other examples of risk change trend (SR-c) graphs. FIG. 12A is a superimposed graph that simultaneously displaying SR-c graphs of three pieces of medical data A to C. FIG. 12B is an SR-c graph generated on the basis of all plots of the superimposed graph shown in FIG. 12A. In this SR-c graph, plots with little risk changes are concentrated near the vertical axis, making it difficult to ascertain a risk change trend.

On the other hand, the SR-c graph shown in FIG. 12C is an SR-c graph generated by performing moving average processing on each piece of medical data of the superimposed graph shown in FIG. 12A. By performing such moving average processing, the risk change trend becomes easier to ascertain, and thus the visibility can be improved.

Further, the SR-c graph shown in FIG. 12D is an SR-c graph generated by performing moving average processing and thinning processing on each piece of medical data of the superimposed graph shown in FIG. 12A. By performing such moving average processing and thinning processing, the risk change trend becomes further easier to ascertain, and thus the visibility can be improved.

Although the visibility of the SR-c graph can be improved by arbitrary processing (for example, moving average processing and thinning processing) as described above, association with v-t graphs and SR-t graphs is simply lost. For example, time information is lost from an SR-c graph by thinning out vertices. As a result, the doctor may misunderstand changes in risk over time. Accordingly, the original vertices of an SR-c graph are generated at corresponding locations on the curves of the processed SR-c graph as follows.

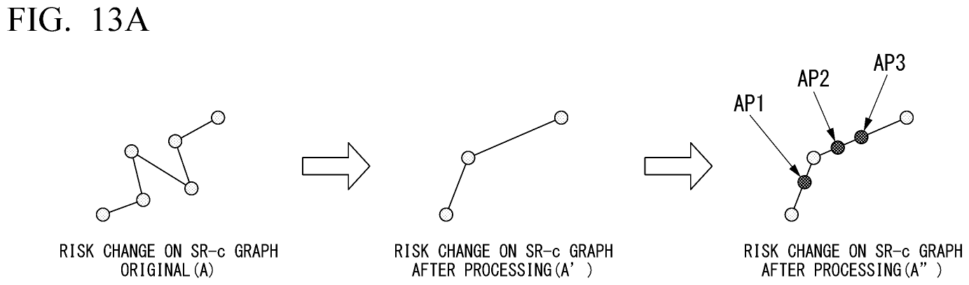

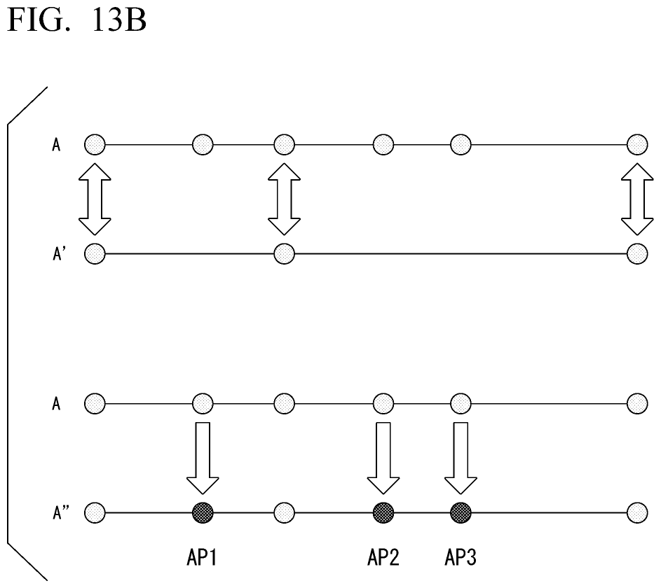

FIGS. 13A and 13B are diagrams for describing vertex interpolation processing of a risk change trend (SR-c) graph. As shown in FIG. 13A, when arbitrary processing (for example, moving average processing and thinning processing) is performed on vertices (original state (A)) indicating risk change on the SR-c graph, a state (A′) after processing in which the number of vertices is reduced from the original state (A) is generated. A state (A″) after processing is generated by performing vertex interpolation processing on the state after processing (A′). At the time of performing vertex interpolation processing, as shown in FIG. 13B, DP matching according to dynamic programming (DP), for example, is performed between the original state (A) and the state (A′) after processing on the basis of information such as the risk value, the amount of risk change, and time held by each vertex, to perform flexible association in one-dimensional spaces. Depending on a processing method, unique association can be achieved (for example, a case in which time has not been processed). In addition, vertices (AP1, AP2, and AP3) are generated in places where there are no vertices in the state (A′) after processing. Vertex intervals are risk change intervals, and thus they are not evenly spaced. If the number of vertices in the state (A′) after processing is greater than that in the original state (A), vertices that are not associated are deleted.

For example, if the vertices in the original state (A) have intervals of one day (a total of 6 vertices indicate data changes for 5 days), there are a total of 3 vertices and data changes for 2 days are seen in the state (A)′ after processing. On the other hand, by performing vertex interpolation processing as described above, it is possible to recognize that data changes for 5 days even in the state after process (A″). Even in the state (A″) after processing, it is still easier to follow risk changes than in the original state (A). This is because the shape of the graph itself does not change even if a vertex is added to the state (A′) after processing.

That is, the generation function 104 (generator) generates display information in the form of a graph in which the horizontal axis represents the amount of changes in the first time-series data and the second time-series data, and the vertical axis represents a standardized risk. The generation function 104 (generator) generates display information indicating, on a graph, processed data obtained by performing predetermined processing on the standardized risk.

According to the second embodiment described above, it is possible to easily check risks of a plurality of types of medical data and support medical decision-making. Accordingly, among the plurality of types of medical data, data to be focused can become obvious. In addition, it is possible to reduce oversight of various risk changes of a patient. Furthermore, it is possible to easily ascertain a risk change trend using a risk change trend (SR-c) graph.

Some or all of the functions of the medical information processing device 1 described above may be realized in the terminal device 3. In this case, the terminal device 3 is an example of a “medical information processing device.”

The embodiments described above can be represented as follows.

A medical information processing device including processing circuitry,
wherein the processing circuitry is configured to:
acquire first time-series data regarding a first parameter and second time-series data regarding a second parameter different from the first parameter;
identify a first risk range regarding the first parameter and a second risk range regarding the second parameter; and
generate display information indicating the first time-series data and the second time-series data in association with each other on a display area in which the first risk range and the second risk range are associated.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising processing circuitry configured to:
acquire first time-series data regarding a first parameter and second time-series data regarding a second parameter different from the first parameter;
identify a first risk range regarding the first parameter and a second risk range regarding the second parameter; and
generate display information indicating the first time-series data and the second time-series data in association with each other on a display area in which the first risk range and the second risk range are associated.

2. The medical information processing device according to claim 1, wherein the processing circuitry is further configured to identify the first risk range and the second risk range on the basis of risk criteria preset depending on types of parameters.

3. The medical information processing device according to claim 1, wherein the processing circuitry is further configured to generate the display information indicating one standardized risk range by normalizing the first risk range and the second risk range.

4. The medical information processing device according to claim 1, wherein the processing circuitry is further configured to calculate a standardized risk and a standardized threshold for each piece of the first time-series data and the second time-series data on the basis of risk criteria preset depending on types of parameters.

5. The medical information processing device according to claim 4, wherein the processing circuitry is further configured to associate the first risk range with the second risk range on the basis of the standardized threshold.

6. The medical information processing device according to claim 4, wherein, when a threshold is set as the risk criteria, the processing circuitry is further configured to set the threshold as the standardized threshold, and calculate, as the standardized risk, values of the first time-series data and the second time-series data, or values obtained by symmetrically transforming the values of the first time-series data and the second time-series data on the basis of the threshold.

7. The medical information processing device according to claim 4, wherein, when a range is set as the risk criteria, the processing circuitry is further configured to calculate the standardized risk on the basis of a midpoint value between an upper limit value and a lower limit value of the range.

8. The medical information processing device according to claim 7, wherein, when two risk ranges are identified for one parameter on the basis of the risk criteria, the processing circuitry is further configured to calculate a value of half a difference between the upper limit value and the lower limit value of the range as the standardized threshold.

9. The medical information processing device according to claim 7, wherein, when one risk range is identified for one parameter on the basis of the risk criteria, the processing circuitry is further configured to calculate the lower limit value of the range as the standardized threshold.

10. The medical information processing device according to claim 4, wherein, when both a threshold and a range are set as the risk criteria for one parameter, the processing circuitry is further configured to identify a quasi-risk range on the basis of the threshold and the range, and calculate a smaller value between the threshold and a lower limit value of a risk range of the one parameter as the standardized threshold.

11. The medical information processing device according to claim 4, wherein the processing circuitry is further configured to generate the display information, which is a graph with a horizontal axis representing time and a vertical axis representing the standardized risk.

12. The medical information processing device according to claim 4, wherein the processing circuitry is further configured to generate the display information, which is a graph with a horizontal axis representing amounts of changes in the first time-series data and the second time-series data and a vertical axis representing the standardized risk.

13. The medical information processing device according to claim 12, wherein the processing circuitry is further configured to generate the display information indicating, on the graph, processed data obtained by performing predetermined processing on the standardized risk.

14. A medical information processing method, using a computer, comprising:
acquiring first time-series data regarding a first parameter and second time-series data regarding a second parameter different from the first parameter;
identifying a first risk range regarding the first parameter and a second risk range regarding the second parameter; and
generating display information indicating the first time-series data and the second time-series data in association with each other on a display area in which the first risk range and the second risk range are associated.

15. A computer-readable non-transitory storage medium storing a program causing a computer to:
acquire first time-series data regarding a first parameter and second time-series data regarding a second parameter different from the first parameter;
identify a first risk range regarding the first parameter and a second risk range regarding the second parameter; and
generate display information indicating the first time-series data and the second time-series data in association with each other on a display area in which the first risk range and the second risk range are associated.
